(19) United States
(12) Patent Application Publication
(10) Pub. No.: US 2014/0023618 A1
(43) Pub. Date: Jan. 23, 2014

(54) SYSTEMS AND METHODS FOR TREATMENT OF ANDROGENETIC SKIN CONDITIONS BY MICROBIAL ORGANISMS

(71) Applicant: APPLIED BIOLOGY, Irvine, CA (US)

(72) Inventors: Andy O. Goren, Newport Beach, CA (US); John McCoy, Downey, CA (US)

(73) Assignee: APPLIED BIOLOGY, Irvine, CA (US)

(21) Appl. No.: 13/843,908

(22) Filed: Mar. 15, 2013

Related U.S. Application Data
(60) Provisional application No. 61/674,819, filed on Jul. 23, 2012.

(51) Int. Cl. A61K 35/74; A61K 31/506; C12N 15/74

(52) U.S. Cl. CPC A61K 35/74; C12N 15/74; A61K 31/506
USPC 424/93.2; 424/93.4

(57) ABSTRACT

A specific naturally occurring or genetically modified bacterial strain can be introduced into the hair follicle of patients suffering from androgenetic alopecia as to reduce the bioavailable extracellular testosterone and hence reduce the effect of androgens on the hair follicle.

### CROSS-REFERENCE TO RELATED APPLICATIONS

This application claims priority to U.S. Provisional Application No. 61/674,819, filed Jul. 23, 2012, entitled “Systems and Methods for treatment of Androgenetic Skin Conditions by Microbial Organisms,” which is incorporated herein by reference in its entirety.

### TECHNICAL FIELD

This subject matter relates systems and methods for treating androgenetic skin conditions such as androgenetic alopecia utilizing microbial organisms.

### BACKGROUND

Various skin and hair conditions such as androgenetic alopecia, hirsutism, acne, and wrinkles are mediated by androgens. Genetic factors interacting with either excess or deficiency of certain androgens result in these conditions.

As an example, androgenic alopecia (AGA), commonly referred to as male or female pattern baldness, is a condition that effects both sexes. AGA leads to the miniaturization and shorting of hair follicles that over time produces shorter and thinner hair. As the name implies, the manifestation of AGA depends on the effects of androgens in genetically predisposed individuals. Testosterone (T) is a steroid hormone secreted in the testes of males and ovaries of females as well as metabolized in tissue. In the hair follicle, peripheral T is converted to the more potent dihydrotestosterone (DHT) by the enzyme 5α-reductase (5αR).

Although both T and DHT can bind to the androgen receptor (AR) and initiate its translocation to the nucleus and the subsequent activation of genes leading to the miniaturization of hair follicles (FIG. 1), DHT has been demonstrated to be the responsible androgen in the progression of AGA; men genetically deficient in 5α-reductase have an absence of AGA and an over expression of 5αR has been observed in the hair follicles of individuals with AGA. The conversion of T to DHT is accomplished by two isoforms of 5αR, type I and type II. 5αRI is the predominate isoform in the human scalp; however, it is found exclusively in the sebaceous gland. In contrast, 5αRII has been localized by immunohistochemistry to be in the innermost layer of the outer root sheath and extending into other regions of the hair follicle. Finasteride, a US FDA approved drug for the treatment of AGA, is a competitive inhibitor of 5αRII. Finasteride reduces the conversion of T to DHT in the root sheath of hair follicles and thus diminishes the activation of AR by the higher affinity androgen, DHT. The efficacy of finasteride in the treatment of AGA is well documented and other competitive inhibitors of 5αRII have also been shown to be effective for the treatment of AGA.

While Finasteride and other anti-androgen drugs provide an effective treatment for AGA, systemic pharmaceutical treatments require frequent administration and inadvertently cause severe side effects. Ideally, a treatment for AGA will not require daily administration and will have little or no side effects. The present disclosure attempts to address these issues by the use of microbial organisms, such as bacteria, in the delivery of effective therapy.

The human microbial flora is extremely diverse. The number of bacteria residing on a person is estimated to outnumber a person's own cells by a ration of 10:1. Under normal circumstances, the majority of these bacteria are symbiotic or commensal; thus, modification of the human bacterial flora provides a unique opportunity to deliver new therapies. Modification of the bacterial flora can be accomplished by probiotic treatment, antibiotic treatment, and genetic alteration of bacterial strains either in combination or independently of the other. For example, Propionibacterium Acnes (P. Acnes) is part of the normal bacterial flora of the hair follicles. By engineering P. Acnes, or another bacterium that is not pathogenic when introduced into the hair follicle, to degrade the extracellular testosterone in the hair follicle, one can effectively reduce intracellular DHT and hence prevent hair loss. A similar technique can be used for the treatment of hirsutism, acne, auxiliary hair reduction, dry skin, and wrinkles by modifying bacteria to deliver or metabolize hormones in the hair or skin.

### BRIEF SUMMARY

The present disclosure relates to various methods. Among these are included a method comprising introducing a microorganism into the hair follicle of a patient suffering from androgenetic alopecia, wherein the microorganism is endogenous to the skin of at least one species of mammal, and wherein the microorganism metabolizes or degrades testosterone.

In another embodiment, there is described a method comprising introducing a microorganism into the hair follicle of a patient suffering from androgenetic alopecia, wherein the microorganism is endogenous to the skin of at least one species of mammal, and wherein the microorganism is genetically modified to secrete a homolog of human sulfotransferase 1A1.

In another embodiment, there is described a method comprising applying a bacterium to acne-diseased human skin, wherein said bacterium is P. acnes in which expression of integration host factor (IHF) and histone-like protein from E. coli strain U93 (HU) have been genetically disrupted.

Various additional embodiments, including additions and modifications to the above embodiments, are described herein.

### BRIEF DESCRIPTION OF THE DRAWINGS

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted, based on this disclosure, in view of the common knowledge within this field.

In the drawings:

FIG. 1 illustrates the testosterone and DHT pathway in AGA.

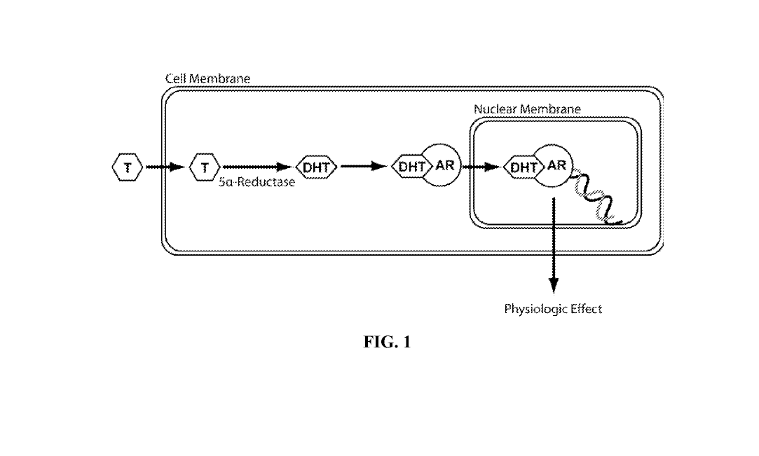

### DETAILED DESCRIPTION

Various example embodiments of the present disclosure are described herein in the context of utilizing microbial organisms in the treatment of androgenetic skin conditions.

Those of ordinary skill in the art will understand that the following detailed description is illustrative only and is not intended to be in any way limiting. Other embodiments of the present disclosure will readily suggest themselves to such skilled persons having the benefit of this disclosure, in light of what is known in the relevant arts, the provision and operation of information systems for such use, and other related areas.

Not all of the routine features of the exemplary implementations described herein are shown and described. In the development of any such actual implementation, numerous implementation-specific decisions must be made in order to achieve the specific goals of the developer, such as compliance with regulatory, safety, social, environmental, health, and business-related constraints, and that these specific goals will vary from one implementation to another and from one developer to another. Moreover, such a developmental effort might be complex and time-consuming, but would nevertheless be a routine undertaking of engineering for those of ordinary skill in the art having the benefit of this disclosure.

Throughout the present disclosure, relevant terms are to be understood consistently with their typical meanings established in the relevant art. However, without limiting the scope of the present disclosure, exemplary clarifications and descriptions of certain terms are provided for relevant terms and concepts as set forth below:

Use of testosterone in any of the examples described herein may be equivalently replaced by the use of estrogen, or any other androgen.

Use of or action on a hair follicle in any of the examples described herein may equivalently be replaced by the use of or action on a sebaceous gland or any location on the skin including the dermal papilla.

Androgenic alopecia (AGA) is the condition commonly referred to as male or female pattern baldness. Reference to AGA in any of the examples described herein may equivalently be replaced by hirsutism, acne, auxiliary hair reduction, dry skin, and wrinkles Use of or reference to bacteria in any of the examples described herein may equivalently be replaced by any microbial organism such as yeast, fungi, or virus.

In one embodiment of the present disclosure, a microorganism, such as a bacterium, may be introduced into the hair follicle of a person suffering from androgenetic alopecia, where the microorganism is endogenous to the skin of a species of mammal, such as a human or the specific person being treated, wherein the microorganism metabolizes or degrades testosterone. Illustrative and non-exclusive examples of such microorganisms may include Camamonas testosterone TA441 (see M. Horinouchi et al., A New Bacterial Steroid Degradation Gene Cluster in Comamonas testosterone TA441 which Consists of Aromatic-Compound Degradation Genes for Seco-Steroids and 3-Ketosteroid Dehydrogenase Genes, Appl. Environ. Microbiol. 2003, 69(8):4421, incorporated herein by reference); Steroidobacter denitrificans DSMZ18526 (Y-L Leu et al., A Novel Tesstosterone Catabolic Pathway in Bacteria, J. Bacteriol. 2011, 193(17):4447, incorporated herein by reference); Corynebacteria sp. G38, G40, G41, B; Brevis sp. CW5, and Micrococcus sp. M-DH2 (see R. Decréau et al., Production of malodorous steroids from androsta-5,16-dienes and androsta-4,16-dienes by Corynebacteria and other human axillary bacteria, J. Steriod Biochem. & Mol. Bio. 2003, 87: 327, incorporated herein by reference).

In another embodiment of the present disclosure, a bacterium may be genetically modified to metabolize or degrade testosterone. Said bacteria may be selected from a strain that is either symbiotic or commensal when introduced to the hair follicle or said bacteria can be altered to be symbiotic or commensal. Bacteria known to be endogenous to the skin and hair follicle include: Actinobacteria, Corynebacteria, Enterococci, Micrococci, Propionibacteria, Staphylococci, Streptococci, Demodex and Malassezia.

In another embodiment of the present disclosure, an ideal vector (strain of bacteria, yeast, or bacteriophage) may be identified for the proposed treatment by sampling skin and/or hair from healthy and diseased tissues and characterizing the metagenomic environment of each state by 16S rRNA phylogenetic markers; thus, selecting the vector that best suites the environment. Alternatives for characterizing the metagenomic environment are known in the art, such as “shotgun” Sanger sequencing, or high-throughput pyrosequencing.

Standard recombinant technologies may then be applied to introduce genes that express proteins known to degrade, metabolize or modify testosterone or other androgens and render them inactive in humans. For example, the CYP19 enzyme from humans is a member of the cytochrome P450 superfamily that converts testosterone to estradiol. Other cytochrome P450 family members have been found in bacteria and shown to hydroxylate testosterone. Any member of the cytochrome P450 superfamily that modifies testosterone to an inactive compound may be used. In addition, genes from the catabolic pathway for testosterone degradation from Comamonas testosteroni, known to react with testosterone, may be used. Steroid sulfotransferases and steroid acyltransferases may also be used. In addition, coryneform bacterial strains that have been cultured from the axillary hair follicles contain catabolic pathways that transform testosterone. These bacteria occur on the skin naturally and may be relocated to the scalp directly.

In one embodiment, a microorganism may be genetically modified by addition of a gene from the catabolic pathway for testosterone degradation from Comamonas testosteroni TA441. Examples of such genes may include ORF 18, ORF 17, tesI, tesH, ORF11, ORF12, or tesDEFG (as described in Horinouchi, 2003, infra., incorporated by reference herein). Also included are any genes from any organism that code for 3-ketosteroid-Δ¹-dehydrogenase (TesH) or 3-ketosteroid-Δ⁴-(5α)-dehydrogenase (TesI) (for example, see id.).

After recombinants have been engineered, strains of bacteria may be grown in media containing testosterone to prove successful cloning. Degradation of testosterone may be measured by a standard ELISA assay using antibodies against testosterone. Alternatively, radio-labeled testosterone may be used to monitor testosterone degradation. Said recombinant bacteria may then be introduced to the hair follicle of the patient in order to treat AGA or other androgenic disorders.

In another embodiment of the present disclosure, the aforementioned genes or gene cassettes can be delivered to endogenous bacteria strains via bacterophage viral transfection of the skin microflora. Bacteriophage cocktails against many of the species commensal on skin will be prepared by standard protocols for phage therapeutics. Said phage therapy is then introduced to the hair follicle of the patient in order to treat AGA. Genes delivered by phage to bacteria strains may become part of the bacterial genome by lateral gene transfer and recombination or may have to be re-introduced at regular intervals.

In yet another embodiment of the present disclosure, a culture of endogenous bacteria from the skin or hair follicle will be selectively bred to achieve the desired result of testosterone degradation. Bacteria cultures may be bred in media containing variable levels of testosterone to apply selective pressure to those bacteria unable to metabolize testosterone. Bacterial strains able survive with testosterone as their only carbon source may then be studied to determine their genetic traits. These strains may be cultured and developed as replacement therapies for AGA. In addition, the activation mechanisms for expression of genes needed for testosterone metabolism will be studied. Any molecules found to activate testosterone metabolic cascades can be considered treatments for AGA in their own right.

In yet another embodiment of the present disclosure, a bacteria sample may be obtained from a single individual or a single population of people and selectively bred to achieve the desired result of testosterone degradation. These bacterial may then be reintroduced back to the host as a personalized replacement therapy.

In yet another embodiment of the present disclosure, a bacteria may be modified to express peptide antigenic epitopes of integration host factor (IHF) and histone-like protein from E. coli strain U93 (HU), both members of the DNABII family of DNA-binding proteins found in biofilms. Expressing these antigens on the skin may prime the immune response and would constitute a vaccine against biofilm.

In yet another embodiment of the present disclosure, an IHF and HU knockout strain of P. acnes may be engineered as a replacement therapy for acne biofilms. This strain can be further engineered to secrete an antibiotic peptide (e.g. lantibiotic) to confer a selective advantage and render it the dominant resident strain of P. acnes on the skin.

In yet another embodiment of the present disclosure, a bacterium may be genetically modified to contain an enzyme or enzymes that modify testosterone such that the metabolite created is a therapeutic drug. For example testosterone could be modified enzymatically to become a 5-alpha-reductase inhibitor similar to finasteride. A new metabolite could also be an androgen receptor antagonist, an inhibitor of a 17-beta-steroid dehydrogenase or an inhibitor prostaglandin D2. The new metabolite may be secreted by the bacteria or introduced when bacteria are lysed naturally in the hair follicle.

In yet another embodiment of the present disclosure, a bacterium may be genetically modified to contain a gene for a therapeutic peptide that will be secreted directly into the hair follicle. The therapeutic peptide can be melinin for the treatment of gray hair.

In yet another embodiment of the present disclosure, a bacterium may be genetically modified to contain an enzyme secreted directly into the hair follicle. The enzyme can be reacted with a second chemical ingredient to achieve a therapeutic treatment for androgenic conditions. For example, homologs of the enzyme sulfotransferase 1A1 (SULT 1A1) can be secreted from bacteria into hair follicles to be used in conjunction with a minoxidil treatment for AGA. SULT 1A1 converts minoxidil into its active form minoxidil sulfate.

In yet another embodiment of the present disclosure, a bacterium may be genetically modified to secrete a peptide or protein that directly binds to testosterone or DHT. This protein can be an androgen receptor homolog or an antibody for testosterone. This protein may compete for testosterone and hence lower the available testosterone to bind to intercellular AR.

In yet another embodiment of the present disclosure, a bacterium may be selected from a strain that is either symbiotic or commensal when introduced to the hair follicle. Said bacteria may then be bread selectively in a medium as to acquire genes required to metabolize or degrade testosterone such as by introducing Comamonas Testeroni bacteria and allowing the bacteria to swap genes.

In yet another embodiment of the present disclosure, a bacterium containing genes for a therapeutic protein or proteins may be further engineered so as to be non-viable on the human scalp without a secondary nutritional supplement. The secondary nutritional supplement may be supplied by a shampoo adjunct to the therapy.

In yet another embodiment of the present disclosure, a non-pathogenic strain of a bacteria normally pathogenic to human skin may be modified to contain genes that give a selective advantage for survival. Said strain may then be used as a replacement therapy for the pathogenic variants of said bacteria. For example, acne has been linked to pathogenic strains of Propionibacterium acnes that secrete chemokines and induce an inflammatory response on skin. A non-pathogenic strain may be isolated from human skin or engineered that does not secrete chemokines The non-pathogenic strain may be further engineered to contain a gene for a antibiotic peptide (e.g. lantibiotic) to be secreted as a selective advantage against other strains of P. acnes.

In yet another embodiment of the present disclosure, a genetically modified version of P. acnes may be engineered to contain an enzyme or enzymes that degrade testosterone. The recombinant P. acnes may be introduced to the skin where it will reduce sebum by lowering testosterone and prevent acne.

In yet another embodiment of the present disclosure, a bacteria or set of bacteria isolated from non-bald scalp follicles of a patient may be applied to the miniaturizing hair follicles in order to establish non-balding bacterial flora. This is commonly referred to as replacement therapy.

In yet another embodiment of the present disclosure, a bacterial virus targeting a specific bacterial strain may be introduced to the hair follicle. Said virus encodes testosterone degrading genes.

In yet another embodiment of the present disclosure, said genetically modified bacteria may be engineered to metabolize or degrade testosterone upon an introduction of an external stimulus such as a specific light, sound, temperature, humidity, pressure. Such mechanism may be accomplished by coupling a bacterial sensor gene to activate the testosterone metabolizing gene.

Exemplary embodiments have been described with reference to specific configurations. The foregoing description of specific embodiments and examples have been presented for the purpose of illustration and description only, and although any invention has been illustrated by certain of the preceding examples, it is not to be construed as being limited thereby.

What is claimed is:

1. A method comprising introducing a microorganism into the hair follicle of a patient suffering from androgenetic alopecia, wherein the microorganism is endogenous to the skin of at least one species of mammal, and wherein the microorganism metabolizes or degrades testosterone.

2. The method of claim 1, wherein the microorganism is selected from the group consisting of Comamonas testosterone, Steroidobacter denitrificans , Cornybacteria, Brevis, and Micrococcus.

3. The method of claim 1, wherein the microorganism is genetically modified to metabolize or degrade testosterone.

4. The method of claim 3, wherein the microorganism is genetically modified to express an enzyme from the cytochrome P450 superfamily.

5. The method of claim 4, wherein the microorganism is genetically modified to express CYP19.

6. The method of claim 3, wherein the microorganism is genetically modified by the addition of a gene from Comamonas testosterone TA441 selected from the group consisting of ORF18, ORF17 , tesl, tesH, ORF11, ORF12, and tesDEFG.

7. The method of claim 3, wherein the microorganism is genetically modified to express genes selected from the group comprising steroid sulfotransferases and steroid acyltransferases.

8. The method of claim 1, wherein the microorganism is endogenous to the hair follicles of the patient.

9. The method of claim 1, wherein the microorganism is a bacterium.

10. The method of claim 9, wherein the bacterium is selected from the group consisting of Actinobacteria, Corynebacteria, Enterococci, Micrococci, Propionibacteria, Staphylococci, Streptococci, Demodex and Malassezia.

11. The method of claim 1, wherein the microorganism is selected by a method comprising:
obtaining a tissue sample from a human comprising one or more hair follicles;
analyzing the tissue sample via environmental genomic sequencing to identify a set of endogenous microorganisms in the sample; and
selecting the microorganism from among said set of endogenous microorganisms.

12. The method of claim 1, wherein the microorganism is genetically modified to express peptide antigenic epitopes of at least one member of the DNABII family of DNA-binding proteins found in biofilms.

13. The method of claim 1, wherein the microorganism is non-viable on the human scalp without a secondary nutritional supplement, further comprising application of a shampoo comprising said secondary nutritional supplement.

14. The method of claim 1, wherein the microorganism metabolizes testosterone through expression of a testosterone metabolizing gene; wherein a bacterial sensor gene is coupled to activate the testosterone metabolizing gene.

15. The method of claim 12, wherein said at least one member is selected from integration host factor (IHF) and histone-like protein from E. coli strain U93 (HU).

16. A method comprising:
introducing a microorganism into the hair follicle of a patient suffering from androgenetic alopecia;
wherein the microorganism is endogenous to the skin of at least one species of mammal; and
wherein the microorganism is genetically modified to secrete sulfotransferase 1A1.

17. The method of claim 16, further comprising treatment of the patient with a topical formula comprising minoxidil.

18. A method comprising applying a bacterium to acne-diseased human skin, wherein said bacterium is P. acnes in which expression of integration host factor (IHF) and histone-like protein from E. coli strain U93 (HU) have been genetically disrupted.

19. The method of claim 18, wherein the bacterium has been further engineered to secrete an antibiotic peptide.
